(19) United States
(12) Patent Application Publication
(10) Pub. No.: US 2009/0062244 A1
(43) Pub. Date: Mar. 5, 2009

(54) PHARMACEUTICAL COMPOSITION

(76) Inventors: Joseph Schwarz, (US); Michael Weisspapir, (US)

Correspondence Address: ALPHARX INC., 168 KONRAD CRESCENT, SUITE 200, MARKHAM, L3R 9T9, CA

(21) Appl. No.: 11/849,652

(22) Filed: Sep. 4, 2007

(51) Int. Cl. A61K 31/573; A61K 31/075; A61K 31/496; A61K 31/4164; A61K 31/5415; A61K 31/18; A61K 31/164; A61P 43/00

(52) U.S. Cl. 514/170; 514/714; 514/254.07; 514/396; 514/226.5; 514/605; 514/628

(57) ABSTRACT

Topical composition for enhanced local and systemic delivery of poorly soluble biologically active compounds, comprises of non-volatile solvent or mixture of solvents to dissolve active component, and moisture absorbent or mixture of sorbents to prevent precipitation or crystallization of insoluble material after application

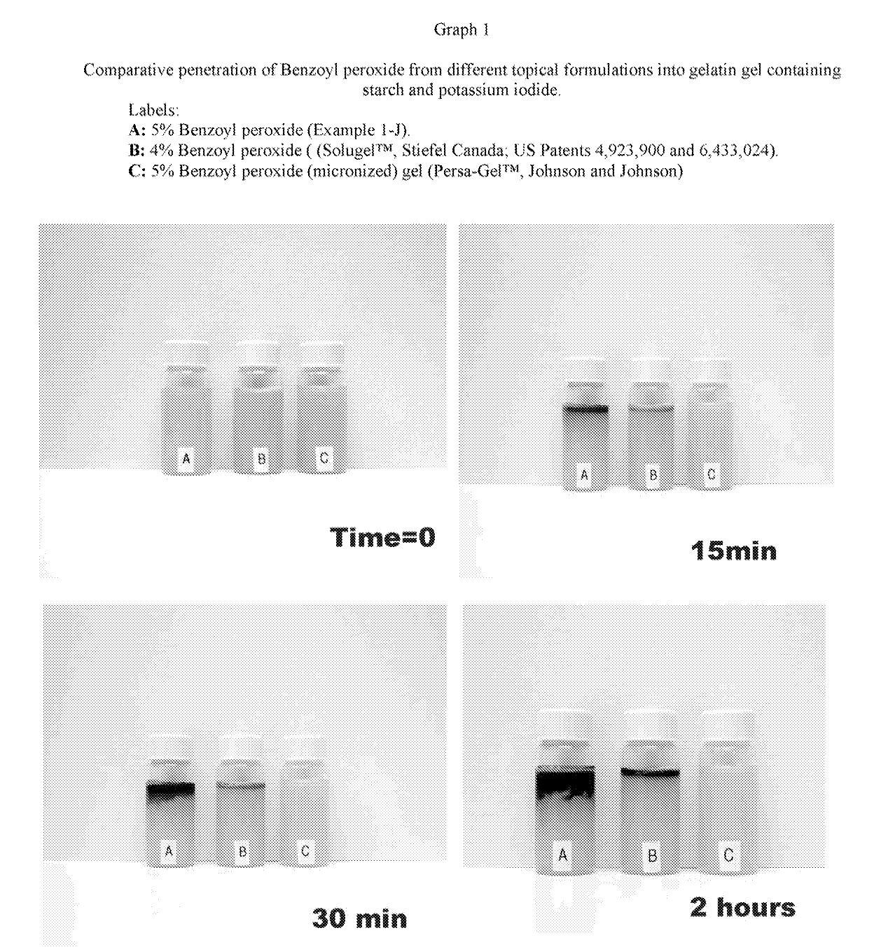

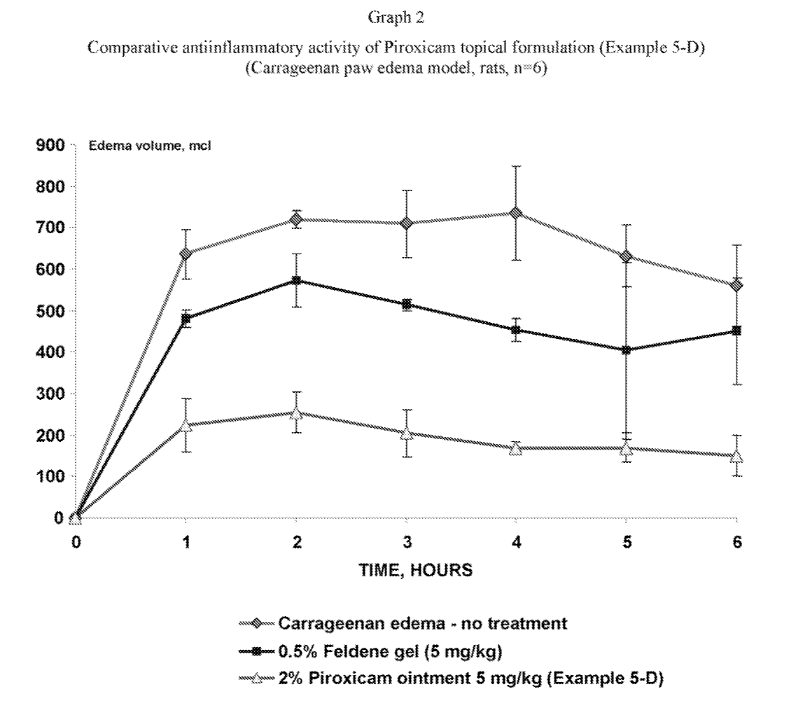

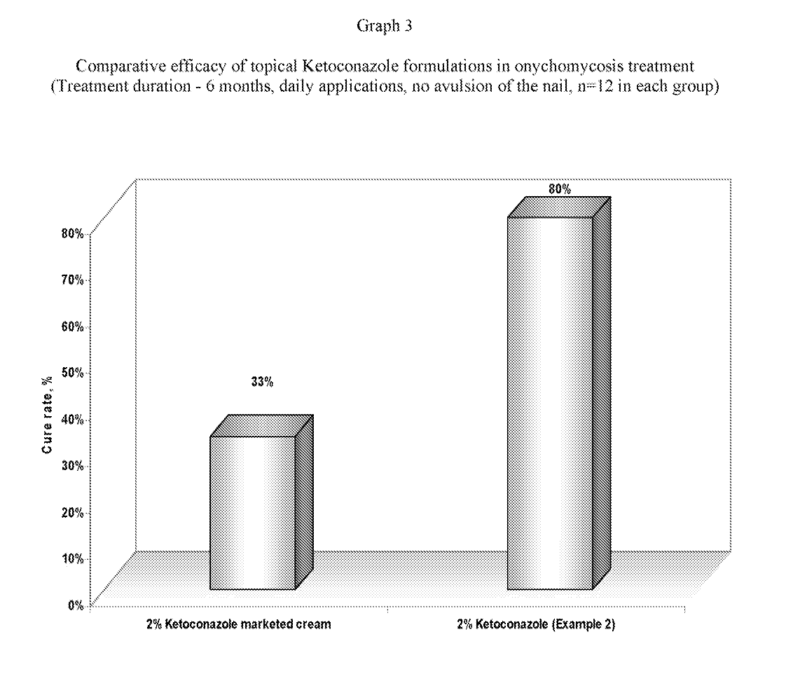

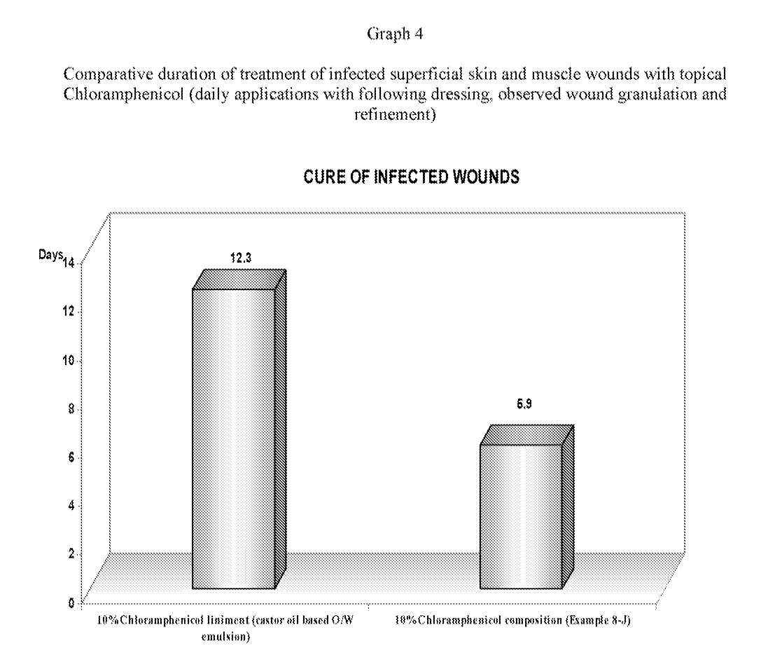

### FIELD OF THE INVENTION

The invention relates to topical formulations which are used for local or systemic topical and transdermal delivery of poorly soluble pharmaceutically active compounds. This invention provides a method for increasing the bioavailability of a pharmaceutical incorporated in the formulation. The inventive topical formulations comprise at least one pharmaceutically active compound and non-aqueous solvent wherein the drug is completely dissolved, along with a moisture absorbent, preventing drug precipitation after contact with skin or mucous membranes.

### BACKGROUND OF INVENTION

It is well known that enhancing the transdermal penetration of topical products is directly proportional to the concentration of drug existing in the formulation in a free dissolved state. According to Fick's Law, flux J is proportional to concentration and the diffusivity coefficient

J=−D*□C/□x

Fick's second law applies to non-steady or continually changing state of diffusion, i.e., when the concentration within the diffusion volume changes with respect to time (e.g., from non-oversaturated solution).

d(C)/dt=D*d²(C)/dx²

Where
J is the diffusion flux as [(amt. of substance)*length⁻²*time⁻¹], e.g., (mol/m²*sec);
D is the diffusion coefficient in dimensions of [length²*time⁻¹], e.g., [m²/sec];
C is the concentration in dimensions of [(amount of substance)*length⁻³], e.g., [mol/m³] and x is the position (distance from diffusion border) [length], e.g., [m]

From these equations it is clear that an increase in the concentration of the free drug in the formulation allows for the increase in the drug flux and thus enhances drug delivery through a diffusional barrier (skin, retina, mucous surfaces, etc.). In order to reach maximal penetration, a drug in a topical formulation incorporated in a maximum concentration, must be completely solubilized.

The solubility of hydrophobic substances in polar solvents varies widely. Several solvents, permitting the high concentration of the dissolved active ingredient, are suitable for epidermal application and can be used in the preparation of topical and transdermal formulations, such as gels, lotions, creams and ointments. DMSO, DMFA, NMP, pyrrolidone-2, dimethylacetamide, glycols-propylene glycol, di ethylene glycol, polyethylene glycol, butylene glycol, hexylene glycol; alcohols-ethyl, isopropyl, butyl alcohol; glycerol and isopropylidene glycerol (Solketal), glycerol formal, tetrafurol, propylene carbonate, ethyl lactate, ethoxydiglycol, dimethyl ether of isosorbide, triacetin and some other polar water miscible solvents have been used for the preparation of topical formulations.

The use of such solvent systems have certain limitations Some solvents cause pronounced skin delipidization (alcohols, N-methylpyrrolidone, dimethylformamide) resulting in irritation, cracking and skin damage. Additionally, the addition of even small amounts of water to such a solution of poorly soluble drugs in these solvents usually causes almost immediate precipitation of the drug. Additionally, when the solvent base formulation is applied to the skin, due to the hypertonicity of the formulation and the miscibility of the solvents with water, the applied formulation absorbs water from the skin and the underlying connective tissues and the drug precipitates on the skin surface. Water penetration causes precipitation thus decreasing of the effective drug concentration in the dissolved stage and reducing drug penetration.

There are many examples of topical formulations containing polar solvents which are capable of achieving the desired solubility of active ingredients in the carrier vehicle. Alcohol- or glycol-based topical gels are widely used for administration of anti-inflammatory agents (e.g. Diclofenac as Voltaren Emulgel, Ketoprofene gel, Ibuprofen gel and spray, Feldene gel. Indomethacin gel and spray, Nimesulide gel, which are available in Europe and Asia), antihistamine (diphenhydramine gel), antiparasitic drugs and cosmetic compositions (see, for example, Guzzo C., et al., U.S. Pat. No. 7,064,108). Patel M. et al. in U.S. Pat. Nos. 6,451,339 and 6,294,192 which describes a vehicle for oral or topical delivery of lipid regulating agents and other poorly soluble hydrophobic agents, based on a combination of a polar solvent and a mixture of hydrophobic and hydrophilic surfactants. De Villez (U.S. Pat. No. 5,086,075) and Popp et al. (U.S. Pat. No. 6,433,024) described formulations of benzoyl peroxide in a mixture of water and polar solvents. Deboeck A. et al. in U.S. Pat. No. 5,036,100 provide a composition of Indomethacin topical lotion, based on dimethylisosorbide and isopropyl alcohol The formulation is non-irritative, but its' anti-inflammatory activity is low when compared to existing topical formulations.

Important to assert that the systemic absorption and transdermal penetration of the incorporated drugs from these formulations is low, despite complete drug solubilization in the vehicle. If a volatile solvent is used, the dissolved drug simply crystallizes on the skin and upper layers of stratum corneum and does not penetrate. For vehicles based on non-volatile water miscible solvents, a fast precipitation of the dissolved hydrophobic compound occurs after the increase of the water content in the vehicle due to absorption of water from body tissues. High levels of solvent provide a high hypertonicity of the vehicle and thus a rapid transfer of the water from body tissues into the vehicle. The solubility of many compounds is extremely sensitive to the water content in the solvent. For example, the solubility of benzoyl peroxide in 95% alcohol at room temperature is several times lower than the solubility in absolute alcohol. A similar dependence on solubility was observed for ketoconazole, itraconazole, diazepam, amphotericin, paclitaxel, etoposide, campthotecin, cyclosporin, ivermectin, diindolylmethane and other poorly soluble hydrophobic compounds. The formation of the crystals of biologically active substances upon contact with the skin surface may not only decrease the penetration, but also produce serious irritation of the epidermis, so it is very important to prevent the formation of precipitates in the formulation.

Soil M. et al. in U.S. Pat. No. 6,991,801 described a topical vehicle for antihelmintic compounds, comprised of drug, solvent, surfactant and a film-forming crystallization inhibitor. Such approach allows the prevention of fast crystallization by the incorporation of formed crystal core seeds into the polymeric film or matrix with high viscosity, thus suppressing the fast growth of the crystals. However, most of the drug remains inside the produced polymeric formation and release of the drug may be seriously diminished by the increase of diffusion resistance through the polymer.

The high concentration of surfactants (20-40% by weight and higher) can also prevent drug crystallization and precipitation in the presence of water by the formation of micelles or an emulsion with incorporated drug and an oily solvent. This approach is used, for example, in U.S. Pat. No. 5,504,068 by Komya, K. et al., for a topical cyclosporin formulation. Nevertheless, in many cases, high concentrations of topically applied surfactants can cause skin irritation.

Oversaturation, as a possible means of keeping a drug in a dissolved state and thus providing a high concentration of drug in the topical vehicle is used in U.S. Pat. No. 5,631,248 by Davis A. et al. After evaporation of a volatile solvent, the remaining mixture of a non-volatile polar solvent with an added mixture of two lipophilic phases of different lipophilicity, combined with a thickener, prevents nucleation and crystallization of the included drug dee to the high viscosity of the formed composition, to allow sufficient time to achieve a high transdermal penetration “in vitro”. A similar approach is realized in U.S. Pat. No. 5,036,100 to Deboeck A. et al., presenting a composition of Indomethacin topical lotion, based on dimethylisosorbide and isopropyl alcohol. The formulation is non-irritative, but its' anti-inflammatory activity is low when compared to existing topical formulations.

The use of volatile organic solvents (alcohol, acetone) in a formulation can cause skin irritation. Skin irritation is also often observed when different skin penetration enhancers, such as Azone (laurocapram), oleic acid, decyl methyl sulfoxide, dodecanol, terpenes, essential oils, etc., are used.

The need for an effective, non-irritating topical vehicle for enhanced transdermal and local topical delivery of poorly soluble drugs remains unfulfilled.

### SUMMARY OF THE INVENTION

A primary object of the invention is to provide a safe and effective formulation for the topical application of biologically active compound(s) by adjusting the solvent system for the particular compound, which will allow the substance to penetrate across the skin barrier with little or no skin response at the site of application and without degrading the chemical structure or bioactivity of the active agent.

Another object of the invention is to provide compositions that are effective for the transdermal delivery of active compounds, where poorly soluble drugs are completely solubilized in a solvent and do not precipitate or crystallize after water absorption during storage or upon application to the skin or mucous membranes.

Another object of the invention is to provide a composition for the transdermal delivery with enhanced penetration through skin and biological tissues.

Still another object of the invention is to provide a versatile solvent base system with water absorbent which is useful for the formulation of topically applied compositions for transdermal administration of a variety of different medicaments with minimal or no modification requirements to achieve a true solution of a medicament and effective, safe, and rapid delivery of the incorporated drug through intact skin or mucous surfaces.

These and other objects of the invention will become clearer upon review of the following more detailed descriptions and specific embodiments and with the aid of the accompanying drawings:
Graph 1. Comparative penetration of Benzoyl peroxide from different topical formulations into gelatin gel containing starch and potassium iodide.
Labels:
A: 5% Benzoyl peroxide (Example 1-J).
B: 4% Benzoyl peroxide in formulation according to U.S. Pat. No. 6,433.024 (Solugel™, Stiefel Canada).
C: 5% Benzoyl peroxide (micronized) gel (Persa-Gel™, Johnson and Johnson)
Graph 2. Comparative antiinflammatory activity of Piroxicam topical formulation (Example 5-D) (Carrageenan paw edema model, rats, n=6)
Graph 3. Comparative onychomycosis treatment with topical Ketoconazole (Example 2-C)
Graph 4. Comparative duration of treatment of infected superficial skin and muscle wounds with topical Chloramphenicol (Example 8-J)

### DETAILED DESCRIPTION OF THE INVENTION

The composition of the present invention provides a means for topically delivering poorly water soluble drugs without crystallization or precipitation of drug during transdermal delivery.

This is exceptionally important since poorly soluble drugs easily precipitate after application on the skin and demonstrate low transdermal flux, compromising specific activity. Additionally, the prevention of drug crystallization and maintaining a high concentration of the active component in the formulation allows to sustain high transdermal flux in accordance with Fick's law.

Surprisingly we found that the addition of the effective water absorbent to a purposely selected solvent or combination of solvents, especially when such sorbent is insoluble or scarcely soluble in these solvents, efficiently prevents drug from precipitation either during storage or when the formulation is applied onto the skin or mucous membranes. Prevention of the drag precipitation is correlated to the moisture absorbent capacity for water, the strength of holding of the absorbed moisture and rapidity of the sorption. The most efficient sorbents are found among inorganic dry sorbents, such as activated zeolites, powdered molecular sieves (SYLOSIV®), dried silicon oxides, calcium silicates, magnesium and aluminium silicates (Talc, Neusilin™, Huberderm™, Hubersorb™), highly dispersed calcium phosphates (Di-Tab®, Tri-Tab®). Chemical absorbents, such as anhydrous sulfates of calcium, magnesium or sodium, are not so efficient due to significant solubility in the solvent system and its' poor compatibility with many active compounds and poor physical stability of the formulation. Additionally, water absorption by these compounds leads to the formation of crystallic hydrates and changes the shape and size of the sorbent particles, which may cause irritation of the skin. Insoluble sorbents, remaining in the same physical state, are found to be more efficient.

Beside inorganic sorbents a high protective behavior against precipitation, caused by moisture, was also demonstrated with insoluble and swell able polymeric sorbents—starch and its derivatives, such as corn, potato, tapioca and rice starch, starch octenylsuccinate (Dry-Flo™), Solanace™, Amaze™ XT; dry cellulose excipients of different types (Avicel™, Vivapur™, milled cellulose Emcocell™, cellulose fibers, lint and fabric), cross-linked polyvinylpyrrolidone (Plasdone™ XL, Crosspovidone™) or slightly cross-linked poly acrylic acid of high molecular weight (Carbopol®)

Unexpectedly it was found that the most efficient protection is provided by moisture absorbents, insoluble or poorly soluble in the solvent mixture of the formulation. Better results were obtained with compounds, solid at room temperature, i.e., having melting point higher than 25° C. Amphiphilic compounds, capable of absorbing and retaining significant amounts of moisture and partially soluble in the solvents system, such as sterols (e.g., cholesterol, lanolin alcohol), long chain alcohols (stearyl alcohol, glyceryl monostearate, glycol palmitate, phosphatidylcholine, hydrogenated lecithin, sucrose tristearate) are also suitable, but somewhat less efficient in preventing crystallization and precipitation of the solubilized drug.

Hygroscopic liquid compounds, such as glycerin, capable of absorbing large quantities of water, are not as efficient in prevention of crystallization, since they are soluble in the composition. Similarly, hydrophilic polymers, soluble in the used solvent mixtures, such as hydroxyethyl-cellulose or low molecular weight polyvinylpyrrolidone, also showed low protection from crystal nucleation.

Another group of hygroscopic solid compounds, polyols and sugars, such as mannitol or sorbitol also can be useful in moisture absorption, but may leave a sticky feeling on the skin if the formulation is not completely absorbed. Polysaccharide based polymers and gums, insoluble in the solvent mixture, such as carboxymethylcellulose, dextran, dextrin, alginic acid and it's esters, carrageenan, xanthan gum, etc. better perform as moisture absorbents. As well solid polyethylene glycols polyethylene oxides and some POE-based solid non-ionic surfactants can be added to a composition as efficient moisture scavengers.

The formulations of the present, invention are semisolid systems, wherein the active component is completely solubilized in the solvent, and effective moisture absorbent is evenly dispersed in the formulation. Microscopic investigation reveals no crystals of the active component.

### EXAMPLE 1

### Benzoyl Peroxide

### Preparation Method:

All components of Part A, Part B and BHT (see table 1) are combined and slowly heated to 45-55° C. with mixing. After complete liquefying the obtained mixture all amount of benzoyl peroxide are slowly added. The composition is mixed until benzoyl peroxide is completely dissolved, then dry absorbent is gradually added and carefully dispersed using appropriate mixer while cooling. Cooling and mixing are carried out until the system reaches the required smooth consistency, and the obtained semisolid composition is packaged into tightly closed containers.

TABLE 1
Compositions of topical semisolid formulation of Benzoyl peroxide

| | 1-A | 1-B | 1-C | 1-D | 1-E | 1-F | 1-G | 1-H | 1-J |
|---|---|---|---|---|---|---|---|---|---|
| BzO2 (calc. as dry base) | 5 | 5 | 5 | 5 | 5 | 5 | 5 | 5 | 5 |
| Part A | | | | | | | | | |
| DMIS | 24 | 24 | 22 | 15 | 22 | 24 | | 24 | 25 |
| Transcutol | 20 | 20 | 18 | 36 | 18 | 20 | 45 | 20 | 22 |
| Part B | | | | | | | | | |
| Solid polyethyleneglycol | 36 | 36 | 6 | 6 | 8 | 8 | 8 | 6 | 8 |
| Hydrogenated lanolin POE | | | 24 | | | 26 | 10 | 28 | 25 |
| Sucrose stearate | | | | 18 | | | | | |
| Cetostearyl alcohol | | | | | 10 | | | | |
| Glyceryl monostearate | | | | 8 | | | | | |
| Cholesterol | | | | | | | 5 | | |
| Cab-O-Sil | 12 | 10 | | | | | 10 | | 5 |
| Zeolite powder (SYLOSIV ™) | | | | | 10 | | | | |
| Ca Silicate Huberderm ™ 1000 | | | | 10 | | 2 | | 2 | |
| Plasdone ™ XL-10 | | | | | | 2 | 4 | 2 | |
| Corn starch | | 1.8 | | | | | | | |
| Amaze ™ XT (starch deriv.) | | | | | 24 | | | | |
| Starch Dry-Flo ™ AF | | | 22 | | | 10 | | 10 | 5 |
| MC cellulose Avicel ™ PH- | | | | | | | 10 | | |
| 103 | | | | | | | | | |
| Butylated hydroxytoluene | 0.2 | 0.2 | 0.2 | 0.2 | 0.2 | 0.2 | 0.2 | 0.2 | 0.2 |
| Physical stability | unstable | stable | stable | unstable | stable | stable | unstable | stable | stable |
| (3 months, 30° C., 75% RH) | | | | | | | | | |
| Penetration into rigid gel stake, | 6 | 7 | 9 | 8 | 6 | 10 | 10 | 12 | 13 |
| mm from surface at 2 hours | | | | | | | | | |

### EXAMPLE 2

### Ketoconazole 2% Semisolid Topical Formulations

All components (Ketoconazole, solvent(s), surfactant, BHA and polyethylene glycol base) excluding triethanolamine are combined and slowly heated to 55-65° C. with mixing. After melting and complete dissolving of Ketoconazole in the obtained mixture dry absorbent is gradually added and carefully dispersed using appropriate mixer while cooling. Triethanol amine is added and cooling and mixing are carried out until the system reaches required smooth consistency. Obtained semisolid composition is packaged into tightly closed containers.

TABLE 2
Compositions of topical semisolid formulations of Ketoconazole

| | 2-A | 2-B | 2-C | 2-D | 2-E | 2-F | 2-G | 2-H | 2-J |
|---|---|---|---|---|---|---|---|---|---|
| Ketoconazole | 2.0 | 2.0 | 2.0 | 2.0 | 2.0 | 2.0 | 2.0 | 2.0 | 2.0 |
| Solvents | | | | | | | | | |
| Dimethylisosorbide | 15.0 | 15.0 | 10.0 | | | | | | |
| Ethoxydiglicol (Transcutol ™_P) | 15.0 | 15.0 | 20.0 | 20.0 | 20.0 | 20.0 | 20.0 | 20.0 | 20.0 |
| Ethyl lactate | | | | 10.0 | | | | | |
| N-Methylpyrrolidone (Pharmasolv ™) | | | | | | | 10.0 | | |
| Dimethylformamide | | | | | | | | 10.0 | 10.0 |
| Propylene glycol | | | | | 10.0 | | | | |
| Pyrrolidone-2 (Soluphor ™) | | | | | | 10.0 | | | |
| Surfactants | | | | | | | | | |
| Lipolan ™ (Hydrogenated POE lanolin) | 25.0 | 12.5 | 12.5 | | 12.5 | 12.5 | 12.5 | 8.5 | 6.0 |
| PEG 20-stearate | | | | 15 | | | | | |
| Moisture absorbents | | | | | | | | | |
| Aerosil ™ 200 | | | | | | | 5.0 | | |
| Sylosiv ™ (powdered mol. sieves) | | | | | | | | 8.0 | |
| Neusilin ™ UFL2 | | | | 12 | | | | | 12.0 |
| Sipernat ™ 500LS | | | | | | 10.0 | | | |
| Solanace ™ (Starch derivative) | | | | | | | 20.0 | | |
| Avicel ™ PH-103 (Microcryst. cellulose) | | | | 5 | 20.0 | 5.0 | | | |
| Starch DryFlo (Starch Octenylsuccinate) | 10.0 | 15.0 | 14.0 | | | | | 12.0 | 12.0 |
| Huberderm ™ 1000 (Ca Silicate anhyd.) | 1.0 | 1.0 | 1.0 | | | | 1.0 | | |
| Plasdone ™ XL-10 | | 1.0 | 1.0 | | | 4.0 | | | |
| Other excipients | | | | | | | | | |
| Polyethylene glycol base USP/NF | 30.0 | 37.5 | 37.5 | 37.5 | 35.0 | 35.0 | 30.0 | 37.5 | 37.5 |
| Butylated hydroxyanisole (BHA) | 0.1 | 0.1 | 0.1 | 0.1 | 0.1 | 0.1 | 0.1 | 0.1 | 0.1 |
| Triethanolamine USP/NF | 0.5 | 0.5 | 0.5 | 0.5 | 0.5 | 0.5 | 0.5 | 0.5 | 0.5 |
| Physical stability | stable | stable | stable | unstab | unstab | stable | stable | stable | stable |
| (6 months, 30° C., 75% RH) | | | | | | | | | |

### EXAMPLE 3

### Itraconazole Semisolid Topical Formulations

Samples are prepared similarly to method described in Example 2, but the composition is heated to 65-75° C. at first step, and triethanolamine is replaced with oleic, succinic, benzoic acid or cetylphosphate in some samples

| | 3-A | 3-B | 3-C | 3-D | 3-E | 3-F | 3-G | 3-H | 3-J |
|---|---|---|---|---|---|---|---|---|---|
| Itraconazole | 1.0 | 1.0 | 1.0 | 1.0 | 1.0 | 1.0 | 1.5 | 1.5 | 2 |
| Solvents | | | | | | | | | |
| Dimethylisosorbide | 30 | 25 | 10 | | | | | | 15 |
| Ethoxydiglicol (Transcutol ™_P) | 15 | 20 | 30 | 20 | | 20 | 20 | 20 | |
| N-Methylpyrrolidone (Pharmasolv ™) | | | | 10 | 15 | | | | |
| Pyrrolidone-2 (Soluphor ™) | | | | | | | 15 | | 20 |
| Ethyl lactate | | | | | 15 | 10 | | 10 | |
| Surfactants | | | | | | | | | |
| Ethoxylated cholesterol (Solulan ™) | 22.5 | 12.5 | 12.5 | 10 | 12.5 | 12.5 | 12.5 | | 12.5 |
| Brij ® 78P (Steareth-20) | | | | | | | | 12.5 | |
| Moisture absorbents | | | | | | | | | |
| Cab-O-Sil ™ M5 | 10 | | | | | | 5 | | |
| Sipernat ™ 22LS | | | 10 | | | | | 8 | |
| Neusilin ™ US2 | | | | 12 | | | | | 10 |
| Huberderm ™ 1000 (Ca Silicate anhydrous) | | | | | 5 | 10 | | | |
| Starch DryFlo ™ (Starch Octenylsuccinate) | | 15 | | | | | 15 | | 10 |
| Avicel ™ PH-103 (Microcryst. cellulose) | | | | 10 | 15 | | | | |
| Plasdone ™ XL-10 | 10 | 5 | 10 | | | 10 | | 12 | |
| Other excipients | | | | | | | | | |
| Polyethylene glycol MW 4000 | 10 | 20 | 25 | 35 | 35 | 35 | 30 | 35 | 30 |
| Cetylphosphate | | | | 1.0 | | | | | |
| Sorbic acid | | | | | 0.5 | | | | |
| Oleic acid | | | | | | 0.5 | | | |
| Benzoic acid | | | | | | | 0.5 | | |
| Succinic acid | | | | | | | | 0.5 | |
| Physical stability (6 months, 30° C., 75% RH) | stable | stable | unstab | unstab | unstab | stable | stable | unstab | stable |

### EXAMPLE 4

### Clotrimazole 5% Semisolid Topical Formulations

Samples are prepared similarly to method described in Example 2.

| | 4-A | 4-B | 4-C | 4-D | 4-E | 4-F | 4-G | 4-H | 4-J |
|---|---|---|---|---|---|---|---|---|---|
| Clotrimazole | 5.0 | 5.0 | 5.0 | 5.0 | 5.0 | 5.0 | 5.0 | 5.0 | 5.0 |
| Solvents | | | | | | | | | |
| Dimethylisosorbide | 15 | 20 | 10 | | 15 | 25 | | | 15 |
| Ethoxydiglicol (Transcutol ™_P) | 15 | 20 | 20 | 20 | | | 20 | 20 | |
| N-Methylpyrrolidone (Pharmasolv ™) | | | | 10 | | | | | |
| Pyrrolidone-2 (Soluphor ™) | | | | | 15 | | 15 | | 20 |
| | | | | | | | | 10 | |
| Surfactants | | | | | | | | | |
| PEG-20 stearate | 20 | 12.5 | | 10 | 12.5 | 12.5 | 12.5 | | 12.5 |
| Sucrose stearate | | | 15 | | | | | 12 | |
| Moisture absorbents | | | | | | | | | |
| Syloid 244 | 10 | 12 | | | | | 5 | | |
| Sipernat ™ 22 | | | 10 | | | | | 8 | |
| Aerosil ™ 200 | | | | 10 | | | | | 8 |
| Huberderm ™ 1000 (Ca Silicate anhydrous) | | 5 | | | 2 | 10 | | | |
| Tapioca starch | | | | | | | 12 | | 10 |
| Alginic acid USP | 5 | | | 10 | 15 | | | | |
| Starch DryFlo ™ (Starch Octenylsuccinate) | | | | | | 12 | | 10 | |
| Glygol monostearate (Peleol ™) | | | 10 | | | | | | |
| Other excipients | | | | | | | | | |
| Polyethylene glycol PEG-4000 | 30 | 25 | 30 | 35 | 35 | 35 | 30 | 35 | 30 |
| Butylated hydroxytoluene (BHT) | 0.1 | 0.1 | 0.1 | 0.1 | 0.1 | 0.1 | 0.1 | 0.1 | 0.1 |
| Physical stability (6 months, 30° C., 75% RH) | stable | stable | stable | stable | stable | stable | stable | stable | stable |

### EXAMPLE 5

### Piroxicam Semisolid Topical Formulations

Samples are prepared similarly to method described in Example 2.

| | 5-A | 5-B | 5-C | 5-D | 5-E | 5-F | 5-G | 5-H | 5-J |
|---|---|---|---|---|---|---|---|---|---|
| Piroxicam | 1.5 | 1.5 | 1.5 | 1.5 | 1.5 | 1.5 | 1.5 | 1.5 | 2.5 |
| Solvents | | | | | | | | | |
| Dimethylisosorbide | 15 | 15 | 15 | | 15 | 20 | | | |
| Ethoxydiglicol (Transcutol ™_P) | 15 | 15 | 15 | 15 | | | | 20 | 20 |
| N-Methylpyrrolidone (Pharmasolv ™) | | | | 15 | | | 15 | | |
| Pyrrolidone-2 (Soluphor ™) | | | | | 15 | | 15 | | 15 |
| | | | | | | | | 10 | |
| Surfactants | | | | | | | | | |
| Lipolan | 15 | 22.5 | | 12.5 | 12.5 | | 12.5 | | 12.5 |
| Pluronic F-68 | | | 15 | | | 22.5 | | 12 | |
| Moisture absorbents | | | | | | | | | |
| Aerosil 200 | | 10 | | | | 8 | 5 | | |
| Sipernat ™ 500LS | 10 | | 10 | | | | | 10 | |
| Sylosiv | | | | | 10 | | | | 5 |
| Huberderm ™ 1000 (Ca Silicate anhyd.) | | | | 10 | | | | | |
| Corn starch | | | | | | | 20 | | 10 |
| Xanthan gum | 10 | 10 | | 10 | | | | | |
| Starch DryFlo (Starch Octenylsuccinate) | | | | | 10 | 12 | | 10 | |
| Carboxymethylcellulose | | | 12 | | | | | | |
| Other excipients | | | | | | | | | |
| Polyethylene glycol PEG-4000 | 30 | 25 | 30 | 35 | | 35 | 30 | 35 | 25 |
| Glyceryl monostearate | | | | | 35 | | | | 10 |
| Physical stability (6 months, 30° C., 75% RH) | stable | stable | stable | stable | stable | stable | stable | stable | stable |

### EXAMPLE 6

### Nimesulide 5% Semisolid Topical Formulations

Samples are prepared similarly to method described in Example 2.

| | 6-A | 6-B | 6-C | 6-D | 6-E | 6-F | 6-G | 6-H | 6-J |
|---|---|---|---|---|---|---|---|---|---|
| Nimesulide | 5.0 | 5.0 | 5.0 | 5.0 | 5.0 | 5.0 | 5.0 | 5.0 | 5.0 |
| Solvents | | | | | | | | | |
| Dimethylisosorbide | 18 | 20 | 15 | | 15 | 20 | | | |
| Ethoxydiglicol (Transcutol ™_P) | 18 | 20 | 20 | 18 | | | | 20 | 20 |
| N-Methylpyrrolidone (Pharmasolv ™) | | | | 18 | | | 20 | | |
| Pyrrolidone-2 (Soluphor ™) | | | | | 20 | | 15 | | 15 |
| | | | | | | | | 10 | |
| Surfactants | | | | | | | | | |
| Lipopeg-39S | 20 | 18 | | 18 | 20 | | 18 | | 10 |
| Cremophor RH-60 | | | 15 | | | 20 | | 20 | |
| Moisture absorbents | | | | | | | | | |
| Cab-O-Sil ® M5 | 10 | | | | | 8 | 5 | | |
| Neusilin US2 | | 12 | 8 | | | | | 10 | |
| Dicalcium phosphate anhydrous (A-Tab ®) | | | | | 14 | | | | 5 |
| Hubersorb ™ 600 (Ca Silicate anhydrous) | 4 | | | 10 | | | | | |
| Calcium sulfate hydrous (micronized) | | | | | | | 16 | | 10 |
| Cholesterol | 5 | | | | | | | | |
| Microcrystalline cellulose Avicel ® PH-103 | | | | | | 16 | | 10 | |
| Avicel ® RC-581 | | | 12 | | 10 | | | | |
| Other excipients | | | | | | | | | |
| Polyethylene glycol PEG-4000 | 20 | 25 | 25 | 30 | | 30 | 20 | 25 | 25 |
| Glyceryl monostearate | | | | | 15 | | | | 10 |
| Physical stability (6 months, 30° C., 75% RH) | stable | stable | stable | stable | stable | stable | stable | stable | stable |

### EXAMPLE 7

### Prednisolone and Prednisolone Acetate 1% Semisolid Topical Formulations

Samples are prepared similarly to method described in Example 2.

| | 7-A | 7-B | 7-C | 7-D | 7-E | 7-F | 7-G | 7-H | 7-J |
|---|---|---|---|---|---|---|---|---|---|
| Prednisolone | 1 | 1 | 1 | 1 | 1 | | | | |
| Prednisolone acetate | | | | | | 1 | 1 | 1 | 1 |
| Solvents | | | | | | | | | |
| Dimethylisosorbide | 15 | 20 | 15 | | | 15 | 18 | 20 | |
| Ethoxydiglicol (Transcutol ™_P) | 15 | 10 | 20 | 18 | 10 | | | | 20 |
| N-Methylpyrrolidone (Pharmasolv ™) | | | | 18 | | 10 | 18 | | |
| Pyrrolidone-2 (Soluphor ™) | | | | | 22 | | | | 15 |
| Propylene glycol | | | | | | | | 10 | |
| Surfactants | | | | | | | | | |
| Lipolan ™ | 22 | 18 | | 18 | 20 | 12 | 18 | | 12 |
| Polyglycerylstearate | | | 22 | | | 10 | | 22 | |
| Moisture absorbents | | | | | | | | | |
| Aerosil 200 | 8 | | | | | 8 | 10 | | |
| Sipernat 22LS | | 12 | 10 | | | | | 12 | 8 |
| Sylosiv | | | | 5 | 8 | | | | |
| Huberderm ™ 1000 (Ca Silicate anhydrous) | | | | 10 | | | | | |
| Sodium starch glycolate (Ac-Di-Sol) | | | | | | | 10 | | 8 |
| Powdered cellulose (Arbocel ™ M80) | 14 | | | | | 20 | | | |
| Carbopol ® 934F | | 10 | | | | | | 10 | |
| Kollidon ® CL-M (Crosspovidone micronized) | | 5 | 6 | | 8 | | | | |
| Other excipients | | | | | | | | | |
| Polyethylene glycol PEG-4000 | | | 25 | 30 | 30 | | 25 | 25 | 25 |
| Cetostearyl alcohol | 25 | 24 | | | | 24 | | | 10 |
| Physical stability (6 months, 30° C., 75% RH) | stable | stable | stable | stable | stable | stable | stable | unstable | stable |

### EXAMPLE 8

### Choramphenicol Semisolid Topical Formulations

Samples are prepared similarly to method described in Example 2.

| | 8-A | 8-B | 8-C | 8-D | 8-E | 8-F | 8-G | 8-H | 8-J |
|---|---|---|---|---|---|---|---|---|---|
| Chloramphenicol | 5 | 5 | 5 | 10 | 10 | 10 | 10 | 10 | 10 |
| Solvents | | | | | | | | | |
| Dimethylisosorbide | 20 | 10 | 15 | | | 15 | 18 | 20 | 20 |
| Ethoxydiglicol (Transcutol ™_P) | 10 | 20 | 20 | 18 | 20 | | | | 20 |
| N-Methylpyrrolidone (Pharmasolv ™) | | | | 10 | | 10 | 18 | | |
| Pyrrolidone-2 (Soluphor ™) | | | | | 10 | | | | |
| Propylene glycol | | | | | | | | 10 | |
| Surfactants | | | | | | | | | |
| LipoPEG-39 | 16 | 18 | | 18 | 20 | 12 | 18 | | 12 |
| Hydrogenated lanolin POE | | | 22 | | | 10 | | 22 | |
| Moisture absorbents | | | | | | | | | |
| Aerosil 200 | 14 | | 10 | | | 8 | 10 | | 10 |
| Carbopol ™ 934F | | 2 | | 4 | 8 | | | | |
| Huberderm ™ 1000 (Ca Silicate anhydrous) | | 4 | 2 | 10 | | | | | |
| Emcocel ™ SP-15 (dried) | 10 | | | | | 10 | | | |
| DryFlo ™ starch derivative | | 16 | | 10 | 10 | | | 12 | |
| Other excipients | | | | | | | | | |
| Polyethylene glycol PEG-4000 | 25 | 25 | 25 | | | | 25 | 25 | 18 |
| Cetostearyl alcohol | | | | 20 | 22 | 24 | | | 10 |
| Physical stability (6 months, 30° C., 75% RH) | stable | stable | stable | stable | stable | unstab | stable | unstab | stable |

### EXAMPLE 9

Semisolid Topical Formulations with Various Biologically Active Compounds

Samples are prepared similarly to method described in Example 2.

| | 9-A | 9-B | 9-C | 9-D | 9-E | 9-F | 9-G | 9-H | 9-J |
|---|---|---|---|---|---|---|---|---|---|
| Active(s) | | | | | | | | | |
| Amphotericin | 1.5 | | | | | | | | |
| Nystatin | | 1.5 | | | | | | | |
| Indomethacin | | | 5 | | | | | | |
| Diindolylmethane (DIM) | | | | 5 | | | | | |
| Metronidazole | | | | | 5 | | | | |
| Diazepam | | | | | | 2 | | | |
| Acyclovir | | | | | | | 5 | | |
| Glipizide | | | | | | | | 1 | |
| Ketoprofen | | | | | | | | | 10 |
| Solvents | | | | | | | | | |
| Dimethylisosorbide | 20 | 15 | | | | 10 | 15 | 25 | 15 |
| Ethoxydiglicol (Transcutol ™_P) | 15 | | 35 | 20 | 20 | | | | 25 |
| N-Methylpyrrolidone (Pharmasolv ™) | | 15 | | | | | 25 | 15 | |
| Pyrrolidone-2 (Soluphor ™) | | | | | 10 | 20 | | | |
| Ethyl lactate | | | | 20 | | | | | |
| Surfactants | | | | | | | | | |
| PEG40 stearate | 15 | 18 | | 18 | 20 | 12 | 18 | | |
| Ethoxylated cholesterol | | | 22 | | | 10 | | 22 | |
| Sucrose palmitate P1670 (Ryoto ® sugar ester) | | | | | | | | | 12 |
| Moisture absorbents | | | | | | | | | |
| Neusilin ™ UF2 | 8 | | | | | | 10 | | |
| Sipernat ™ 50S | | 8 | 8 | 4 | 8 | 10 | | | 10 |
| Huberderm ™ 1000 (Ca Silicate anhydrous) | | | 2 | 10 | | | 2 | | |
| Chitosan | 20 | | | 12 | | 10 | | | |
| Gum Karaya | | 20 | | | | | | 12 | |
| DryFlo ® starch octenyl succinate | | | | | 15 | | | | |
| Other excipients | | | | | | | | | |
| Polyethylene glycol PEG-4000 | 20 | 22 | 28 | | 22 | 25 | 25 | 25 | 18 |
| Glyceryl monostearate | | | | 10 | | | | | 10 |
| Physical stability (6 months, 30° C., 75% RH) | stable | stable | stable | stable | stable | stable | stable | stable | stable |

### REFERENCES

### U.S. Patents:

1. Guzzo C., et al., U.S. Pat. No. 7,064,108

2. Patel M., et al., U.S. Pat. No. 6,451,338

3. Patel M., et al., U.S. Pat. No. 6,294,192

4. De Villez, et al., U.S. Pat. No. 5,086,075

5. Popp, et al., U.S. Pat. No. 6,433,024

6. Deboeck A., et al., U.S. Pat. No. 5,036,100

1. A water washable anhydrous pharmaceutical composition for the enhanced topical delivery of biologically active poorly water soluble compounds; said composition comprises of;
a) at least one biologically active compound in amounts between 0.01 and 20% by weight presented in dissolved state
b) a water miscible solvent or mixture of solvents as a solubilizer of a biologically active compound
c) a moisture absorbent, insoluble or poorly soluble in the said solvent
d) at least one physiologically acceptable water miscible or water dispersible surfactant in amounts between 0.5 and 60% by weight

2. A composition of claim 1 wherein said biologically active compound is completely solubilized in said solvent and does not precipitate immediately on contact with skin or wet surface

3. A composition of claim 1 wherein said moisture absorbent does not decrease solubility of biologically active hydrophobic compound in composition and does not cause its precipitation during storage

4. A pharmaceutical composition as set forth in claim 1, containing no triglycerides or hydrophobic esters

5. A composition of claim 1 wherein said water miscible solvent is selected from a group of aliphatic alcohols, glycols, propylene glycol, butylene glycol, polyethylene glycols, ethoxydiglycol, isosorbide ethers, propylene carbonate, dimethylsulfoxide (DMSO), dimethylacetamide (DMA), dimethylformamide (DMFA), isopropylidene glycerin (Solketal®), glycerol formal, tetrafurol, dimethylisosorbide, ethyllactate, N-methylpyrrolidone (Pharmasolve®), pyrrolidone-2 (Soluphor®) or a mixture thereof

6. A composition of claim 1 wherein said moisture absorbent is selected from a group of inorganic silicates, phosphates, carbonates, dried silicagels, colloidal, amorphous or granulated silicon dioxide, alumosilicates, zeolites, powdered molecular sieves; polysaccharides, powdered cellulose, cellulose fibers, cellulose lint or fabric; microcrystalline cellulose, starch and starch derivatives, alginic acid and salts thereof, cellulose gum, xanthan gum or acacia gum, crosslinked polyacrylates or polyvinylpyrrolidone, or a mixture thereof

7. A composition as set forth in claim 1 wherein said surfactant is selected from a group of dermatologically acceptable polyethoxylated aliphatic or aromatic derivatives, polyglycerin derivatives, sugar and polyol esters or ethers anionic surfactants, cationic surfactants or a mixture thereof

8. A composition of claim 1 wherein said moisture absorbent comprises of physiologically acceptable hydrophobic or amphiphilic compounds with melting point not less than 25° C., selected from a group of cetearyl or cetostearyl alcohol, polyethylene glycols, ethoxylated esters of aliphatic or aromatic alcohols or sterols, lecithin and phospholipids, alkyl esters of glycerol or alkyl esters of glycols, or a mixture thereof

9. A composition of claim 1 wherein said water miscible solvent comprises of dimethylisosorbide, ethoxydiglycol or a mixture thereof in ratio from 1:10 to 10:1, preferably 1:3 to 3:1, in amounts between 10% and 90% of the composition by weight.

10. A pharmaceutical composition as set forth in claim 1, wherein said compound is selected from a group of non-steroidal antiinflammatory drugs (NSAIDs), steroids, hormones, liposoluble vitamins, prostaglandins, local anesthetics, analgesics, antivirals, antibacterials, antibiotics, antifungals, antimetabolites, cytostatics, antipsoriatics, retinoids, immune suppressors, antihistamines, tranquilizers, pyrethroids, antiparasitics, diindolylmethane, organic acids and benzoyl peroxide.

11. A pharmaceutical composition as set forth in claim 1, which may further contain pharmaceutically acceptable excipients, surfactants, antioxidants, preservatives, stabilizers, sorbents, solvents, rheology modifiers, colorants and fragrances.
